(19) United States
(12) Patent Application Publication
(10) Pub. No.: US 2016/0136282 A1
(43) Pub. Date: May 19, 2016

(54) NOVEL FORMULATION OF CILOSTAZOL, A QUINOLINONE-DERIVATIVE USED FOR ALLEVIATING THE SYMPTOM OF INTERMITTENT CLAUDICATION IN PATIENTS WITH PERIPHERAL VASCULAR DISEASE

(71) Applicant: Genovate Biotechnology Co., Ltd., Hsin-Chu (TW)

(72) Inventors: Fengchu Chen, Hsin-Chu (TW); Yinshan Wang, Hsin-Chu (TW); Yilun Yang, Zhanghua County (TW); Yuying Lin, Xinzhu County (TW)

(21) Appl. No.: 14/546,864

(22) Filed: Nov. 18, 2014

(51) Int. Cl. A61K 47/38; A61K 9/00; A61K 47/26; A61K 31/4709; A61K 9/14

(52) U.S. Cl. CPC A61K 47/38; A61K 31/4709; A61K 9/14; A61K 47/26; A61K 9/00

(57) ABSTRACT

A pharmaceutical composition in solid form containing particulate cilostazol or a salt thereof, a cellulose, a diluent, and a lubricant. The pharmaceutical composition features an in vivo plasma profile for cilostazol of C_{24 h}/Cₘₐₓ>0.25. Also disclosed is a method of preparing the above-described pharmaceutical composition.

### FIELD OF THE INVENTION

The present invention relates to pharmaceutical formulations of cilostazol, a phosphodiesterase inhibitor, more particularly to extended-release dosage forms suitable for administration once daily.

### BACKGROUND OF THE INVENTION

Cilostazol, a selective inhibitor of phosphodiesterase-3, inhibits platelet aggregation and acts as a direct arterial vasodilator. It is commercially available as Pletaal® tablets manufactured by Otsuka Pharmaceutical, the listed indications of which include relieving symptoms of intermittent claudication.

Patients suffering from intermittent claudication easily develop leg pain and limp and cannot walk a long distance without taking a rest. The intensity of the disease can be clinically measured either by initial claudication distance, i.e., the distance a patient can walk before a pain develops, or by absolute claudication distance, i.e., the distance a patient can walk until a rest has to be taken.

Intermittent claudication, a common disease among the elderly, is a clinical manifestation of peripheral vascular disease, often referred to as peripheral artery occlusive disease. Its causes include atherosclerotic lesions and disorders in platelet activation, which result in gradually narrowed arteries and ischemia symptoms. Cilostazol, a phosphodiesterase-3 inhibitor, and its metabolites elevate the concentration of cAMP in blood by blocking its metabolism, leading to therapeutic effects of anti-platelet aggregation and blood vessel expansion.

Cilostazol has been used for treating intermittent claudication. 50 and 100 mg Pletaal® tablets require two administrations per day. They are immediate-release tablets that disintegrate rapidly in the body and can cause serious adverse reactions when the cilostazol concentration in blood rise abruptly. Reported side effects attributable to cilostazol include headache, abnormal stools, diarrhea, dizziness, and palpitations.

There is a need to develop an extended-release form of cilostazol that, upon administration, achieves a more stable cilostazol blood concentration that would contribute to fewer side effects. In addition, an extended-release form can be taken only once per day, thereby facilitating patient compliance.

### SUMMARY OF THE INVENTION

This invention provides a pharmaceutical composition, i.e., an extended-release form of cilostazol, which, unexpectedly, has a higher efficacy and fewer side effects than Pletaal®. As such, it can be administered once daily for treating intermittent claudication.

One aspect of this invention relates to a pharmaceutical composition in solid form that contains particulate cilostazol or a salt thereof, a cellulose, a diluent, and a lubricant. The particulate cilostazol or salt thereof has a 90% particle size in a cumulative particle size distribution of 5-75 μm (preferably, 10-30 μm) and constitutes 15% to 70% (preferably, 25% to 55%) by weight of the pharmaceutical composition. The pharmaceutical composition of this invention features an in vivo plasma profile for cilostazol of C_{24 h}/Cₘₐₓ>0.25 (preferably, C_{24 h}/Cₘₐₓ>0.5).

The cellulose, either water soluble or water insoluble, can be hydroxypropylmethylcellulose (HPMC), hydroxyethyl cellulose, hydroxyethylmethyl cellulose, hydroxypropyl cellulose, ethylcellulose (EC), cellulose acetate phthalate, cellulose acetate, methylcellulose, hypromellose phthalate, or a combination thereof. Among them, HPMC, EC, and a combination thereof are preferred.

On the other hand, the diluent can be calcium carbonate, calcium phosphate, calcium sulfate, dextrates, dextrose, erythritol, fructose, kaolin, lactitol, lactose, mannitol, simethicone, sodium chloride, sorbitol, starch, sucrose, sulfobutylether-β-cyclodextrin, trehalose, xylitol, microcrystalline cellulose, or a combination thereof. Lactose, microcrystalline cellulose, and a combination thereof are preferred.

Examples of the lubricant include calcium stearate, glycerin monostearate, glyceryl behenate, glyceryl palmitostearate, hydrogenated castor oil, hydrogenated vegetable oil, light mineral oil, sodium lauryl sulfate, sodium stearyl fumarate, stearic acid, zinc stearate, sodium benzoate, magnesium stearate, myristic acid, palmitic acid, poloxamer, polyethylene glycol, potassium benzoate, talc, and a combination thereof. Magnesium stearate, stearic acid, and a combination thereof are preferred.

In one embodiment, this pharmaceutical composition contains particulate cilostazol or a salt thereof in the amount of 100 mg. In another embodiment, the amount of particulate cilostazol or a salt thereof is 200 mg.

Another aspect of this invention relates to a method of preparing the above-described pharmaceutical composition. The method includes the following steps: (i) mixing particulate cilostazol or a salt thereof having a 90% particle size in a cumulative particle size distribution of 5 to 75 μm, a first cellulose, a diluent, and water to form a homogenous mixture; (ii) granulating the homogenous mixture to form granules; (iii) heating the granules to form dried granules; (iv) mixing the dried granules, a lubricant, and optionally a second cellulose (i.e., different from the first cellulose) to form a blend; and (v) compressing the blend to form tablets. The pharmaceutical composition thus prepared contains particulate cilostazol or a salt thereof, a first cellulose, a diluent, a lubricant, and optionally a second cellulose.

The first cellulose can be HPMC, hydroxyethyl cellulose, hydroxyethylmethyl cellulose, hydroxypropyl cellulose, ethylcellulose, cellulose acetate phthalate, cellulose acetate, methylcellulose, hypromellose phthalate, or a combination thereof.

The second cellulose can be EC, cellulose acetate phthalate, cellulose acetate, methylcellulose, hypromellose phthalate, or a combination thereof.

Examples of the diluent and the lubricant are enumerated above.

The details of the invention are set forth in the drawings and description below. Other features, objects, and advantages of the invention will be apparent from the description and from the claims.

### BRIEF DESCRIPTION OF THE DRAWINGS

FIG. 1 illustrates exemplary release rate profiles for four representative cilostazol compositions, i.e., Examples 1-4, in accordance with the teachings of this invention.

FIG. 2 illustrates exemplary release rate profiles for three representative cilostazol compositions, i.e., Examples 5-7, in accordance with the teachings of this invention.

FIG. 3 illustrates exemplary release rate profiles for three representative cilostazol compositions, i.e., Examples 8-10, in accordance with the teachings of this invention.

FIG. 4 illustrates exemplary release rate profiles for four representative cilostazol compositions, i.e., Examples 11-14, in accordance with the teachings of this invention.

FIG. 5 illustrates exemplary in vivo plasma profiles for two representative cilostazol compositions, i.e., Examples 15 and 16, in accordance with the teachings of this invention.

### DETAILED DESCRIPTION

The pharmaceutical composition of this invention can be a Pletaal® modified release tablet, which is referred herein as “PMR.” PMR is an extended-release form of cilostazol. By contrast, Pletaal® is an immediate-release cilostazol tablet.

The PMR tablet contains as an active ingredient particulate cilostazol or a salt thereof. Particulation of cilostazol, a drug insoluble in water, enhances its bioavailability. The cumulative particle size distribution of the particulate cilostazol or salt thereof is measured using a Malvern Mastersizer according to the known method described in International Patent Application Publication WO 2007/027612. D(0.9) is defined as the size of 90% of the particles based on the measured cumulative particle size distribution. Likewise, D(0.5) and D(0.1) are defined as the sizes of 50% and 10% of the particles, respectively. D(0.9) of particulate cilostazol or a salt thereof is preferably 10-20 μm and, more preferably, 10-15 μM; D(0.5) is preferably 5-10 μm and, more preferably, 6-8 μm; and D(0.1) is preferably 0.3-1 μm and, more preferably, 0.4-0.7 μm.

This particulate cilostazol or salt thereof constitutes 15% to 70% by weight of the pharmaceutical composition. To maintain the therapeutic effect, a PMR tablet has to contain the active ingredient above a minimal level (e.g., 15 wt %). On the other hand, excessive levels of the active ingredient (e.g., >70 wt %) undermine the extended-release capability of a PMR tablet.

The PMR tablet also contains one or more celluloses, which can be either water-soluble or non-water soluble.

Water-soluble cellulose, when dissolved in water, forms porous hydrophilic colloid matrices so that a slow release of the drug trapped in the colloid matrices is achieved. Examples of this type of cellulose include HPMC, hydroxyethyl cellulose, hydroxyethylmethyl cellulose, and hydroxypropyl cellulose. HPMC having a high viscosity is preferred. For example, at 25° C., a 2% (w/v) HPMC in an aqueous solution can have a viscosity of >10,000 mPa, even >50,000 mPa. The water-soluble cellulose constitutes 1% to 25% (preferably, 2% to 10%) by weight of the pharmaceutical composition.

Non-water-soluble cellulose acts to adjust the dissolution rate of the drug in a pharmaceutical composition. Examples include EC, cellulose acetate phthalate, cellulose acetate, methylcellulose, and hypromellose phthalate. EC having a medium viscosity is preferred. For example, at 25° C., a 5% (w/v) EC in a toluene/ethanol (80:20 w/w) solution can have a viscosity of 12-110 mPa, 18-80 mPa being preferred and 40-52 mPa being more preferred. The non-water-soluble cellulose, if used, constitutes no more than 50% (preferably, 20% to 40%) by weight of the pharmaceutical composition.

The PMR tablet can include, among others, Povidone, that acts as a binder to adjust the dissolution rate of the active ingredient. When Povidone is used, the Povidone products having a molecular weight of 8,000-400,000 are preferred and those having a molecular weight of 30,000-100,000 are more preferred. The diluent constitutes no more than 25% (preferably, 2% to 10%) by weight of the pharmaceutical composition.

In in vitro dissolution tests, the PMR tablet exhibited a profile of zero degree release.

In pharmacokinetics (PK) studies after a single dose in healthy adult human subjects, this tablet exhibited an in vivo plasma profile superior to that of Pletaal®. More specifically, the PMR tablet showed a maximum plasma concentration (Cₘₐₓ) lower than that of Pletaal® and a plasma concentration at the 24th hour (C_{24 h}) greater than that of Pletaal®, when these two tablets were administered each with an equivalent amount of cilostazol. Furthermore, the PMR tablet exhibited a ratio of C_{24 h} to Cₘₐₓ>0.25.

In the above-mentioned PK studies, the PMR tablet exhibited a bioavailability, as expressed conventionally by the area under curve (AUC) of 4600-13400 ng*hr/ml. Moreover, it exhibited a Cₘₐₓ of 280-800 ng/ml and a C_{24 h} of >0.1 μg/ml.

In one embodiment, the PMR tablet has particulate cilostazol or a salt thereof in the amount of 100 mg and features a Cₘₐₓ of 280-660 ng/ml and an AUC of 4600-7900 ng*hr/ml.

In another embodiment, the PMR tablet has particulate cilostazol or a salt thereof in the amount of 200 mg and features a Cₘₐₓ of 470-800 ng/ml and an AUC of 6400-13400 ng*hr/ml.

Described below are exemplary procedures for preparing PMR tablets of this invention.

Table 1 lists four different workflow models based on which a different combination of a cellulose and a diluent are used to prepare a PMR tablet. Take Model 1 for example. First, particulate cilostazol or a salt thereof having a D(0.9) of 5-75 μm, a diluent (not shown in Table 1), e.g., lactose, and HPMC are mixed with water to form a homogenous mixture. Next, the homogenous mixture is granulated to form granules, followed by heat-drying. The dried granules are then mixed with a lubricant (not shown in Table 1), e.g., stearic acid, to form a blend. Finally, the blend is compressed to form tablets.

TABLE 1
Four models for preparing a PMR

| | Model 1 | Model 2 | Model 3 | Model 4 |
|---|---|---|---|---|
| Before granulation | A | A, B | A | A, C |
| After granulation | — | — | C | C |

A: a water-soluble cellulose
B: a binder (e.g., Povidone)
C: a non-water-soluble cellulose

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated by reference in their entirety.

### Examples 1-4

### Extended-Release Tablets of Cilostazol and their In Vitro Dissolution Profiles

PMR Examples 1-4, each containing non-particulate cilostazol in the amount of 100 mg, were prepared from the ingredients shown in Table 2 below following workflow Model 1 described in Table 1.

TABLE 2
Compositions for PMR Examples 1-4.

| | Example | Example | Example | Example |
|---|---|---|---|---|
| Ingredients | 1 (mg) | 2 (mg) | 3 (mg) | 4 (mg) |
| Cilostazol | 100 | 100 | 100 | 100 |
| Lactose anhydrous | 80 | 53 | 73 | 63 |
| HPMC K100M | 13 | 40 | 20 | 30 |
| Stearic acid | 7 | 7 | 7 | 7 |
| Total | 200 | 200 | 200 | 200 |

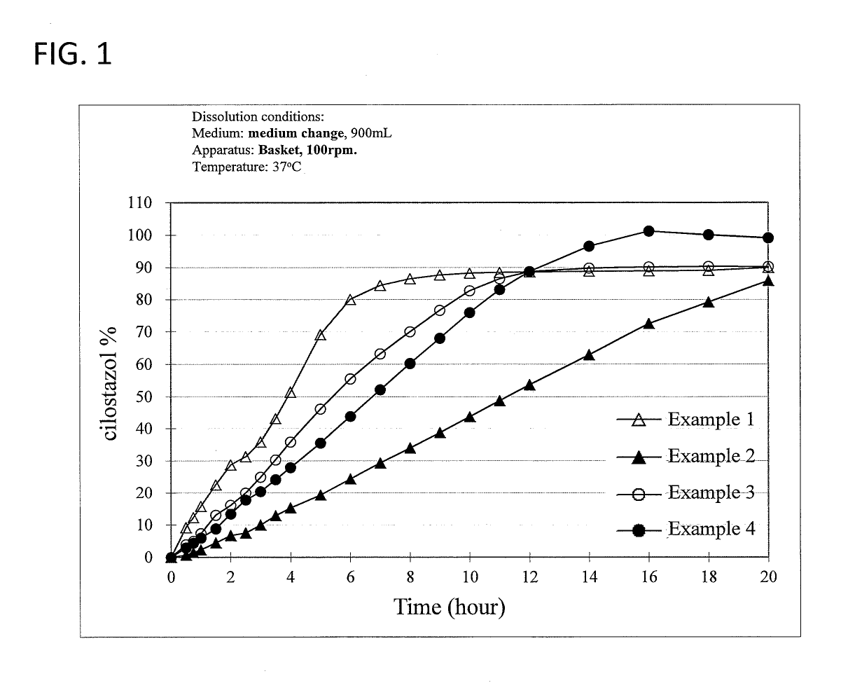

A study was conducted to assess the in vitro dissolution profiles of Examples 1-4. The study was performed according to the procedure described in United States Pharmacopeia (USP36, 2031). More specifically, Examples 1-4 were each placed in a dissolution medium under the temperature of about 37° C. and the dissolution medium was paddled at a speed of about 50 or 100 rpm. Cilostazol concentrations in the dissolution medium were measured at different time intervals. Results are shown in Table 3 below and FIG. 1.

TABLE 3
In vitro dissolution profiles of PMR Examples 1-4.

| | % released Cilostazol | | | |
|---|---|---|---|---|
| Time (hour) | Example 1 | Example 2 | Example 3 | Example 4 |
| 0 | 0 | 0 | 0 | 0 |
| 0.5 | 9.16 | 0.67 | 3.92 | 2.93 |
| 0.75 | 12.25 | 1.48 | 4.97 | 4.42 |
| 1.0 | 15.76 | 2.31 | 7.31 | 5.96 |
| 1.5 | 22.53 | 4.46 | 12.99 | 8.76 |
| 2.0 | 28.74 | 6.70 | 16.08 | 13.34 |
| 2.5 | 31.25 | 7.47 | 19.95 | 17.73 |
| 3 | 35.80 | 9.91 | 24.93 | 20.38 |
| 3.5 | 43.14 | 12.83 | 30.30 | 24.17 |
| 4 | 51.49 | 15.27 | 35.82 | 27.90 |
| 5 | 69.06 | 19.29 | 46.15 | 35.48 |
| 6 | 80.01 | 24.33 | 55.49 | 43.81 |
| 7 | 84.38 | 29.33 | 63.15 | 52.16 |
| 8 | 86.48 | 33.97 | 69.97 | 60.20 |
| 9 | 87.65 | 38.71 | 76.64 | 67.93 |
| 10 | 88.20 | 43.71 | 82.69 | 75.91 |
| 11 | 88.44 | 48.73 | 86.48 | 83.03 |
| 12 | 88.60 | 53.71 | 88.70 | 88.74 |
| 14 | 88.78 | 62.85 | 89.79 | 96.55 |
| 16 | 88.90 | 72.49 | 90.13 | 101.16 |
| 18 | 89.06 | 79.20 | 90.29 | 100.05 |
| 20 | 89.93 | 85.82 | 90.25 | 99.07 |

The results show that Examples 1-4 each exhibited a profile of zero degree release in a dissolution medium.

### Examples 5-7

### Extended-Release Tablets of Cilostazol and their In Vitro Dissolution Profiles

PMR Examples 5-7, each containing particulate cilostazol in the amount of 100 mg, were prepared from the ingredients shown in Table 4 below following workflow Model 1 described in Table 1. Note that, compared to Examples 1-4, Examples 5-7 used particulate cilostazol that has a D(0.9) of 5.1-75.2 μm.

TABLE 4
Compositions for PMR Examples 5-7.

| | Example | Example | Example |
|---|---|---|---|
| Ingredients | 5 (mg) | 6 (mg) | 7 (mg) |
| Cilostazol | 100 D(0.9) | 100 D(0.9) | 100 D(0.9) |
| | 5.1 μm | 13.5 μm | 75.2 μm |
| Lactose anhydrous | 53 | 53 | 53 |
| HPMC K100M | 40 | 40 | 40 |
| Stearic acid | 7 | 7 | 7 |
| Total | 200 | 200 | 200 |

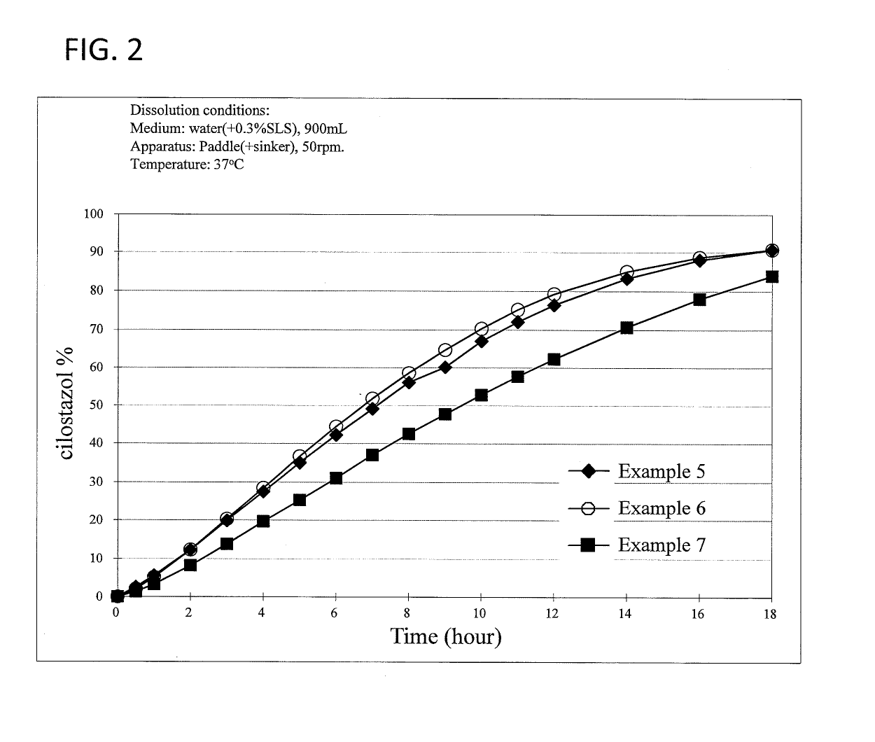

A study was conducted to assess the in vitro dissolution profiles of Examples 5-7. The study was performed according to the procedure described above. Results are shown in Table 5 below and FIG. 2.

TABLE 5
In vitro dissolution profiles of PMR Examples 5-7.

| | % released Cilostazol | | |
|---|---|---|---|
| Time (hour) | Example 5 | Example 6 | Example 7 |
| 0 | 0.00 | 0.00 | 0.00 |
| 0.5 | 2.54 | 2.07 | 1.33 |
| 1 | 5.57 | 5.13 | 3.28 |
| 2 | 12.33 | 12.37 | 8.23 |
| 3 | 19.92 | 20.32 | 13.83 |
| 4 | 27.52 | 28.49 | 19.66 |
| 5 | 35.00 | 36.62 | 25.34 |
| 6 | 42.21 | 44.43 | 31.04 |
| 7 | 49.12 | 51.80 | 36.97 |
| 8 | 56.08 | 58.61 | 42.51 |
| 9 | 60.18 | 64.77 | 47.75 |
| 10 | 67.10 | 70.32 | 52.83 |
| 11 | 72.09 | 75.22 | 57.70 |
| 12 | 76.43 | 79.26 | 62.35 |
| 14 | 83.28 | 85.09 | 70.73 |
| 16 | 88.07 | 88.74 | 78.01 |
| 18 | 90.68 | 90.73 | 83.98 |

The results show that, compared to Examples 1-4, Examples 5-7, which were prepared with particulate cilostazol, exhibited a more consistent profile of zero degree release in a dissolution medium. See FIGS. 1 and 2.

### Examples 8-10

### Extended-Release Tablets of Cilostazol and their In Vitro Dissolution Profiles

PMR Examples 8-10, each containing particulate cilostazol in the amount of 100 mg, were prepared from the ingredients shown in Table 6 below following workflow Model 2 described in Table 1.

TABLE 6
Compositions for PMR Examples 8-10.

| | Example | Example | Example |
|---|---|---|---|
| Ingredients | 8 (mg) | 9 (mg) | 10 (mg) |
| Cilostazol | 100 | 100 | 100 |
| D(0.9) <20 μm | | | |
| Lactose anhydrous | 53 | 53 | 53 |
| HPMC K100M | 40 | 40 | 40 |
| Stearic acid | 7 | 7 | 7 |
| Povidone K30 | 50 | 25 | 10 |
| Total | 250 | 225 | 210 |

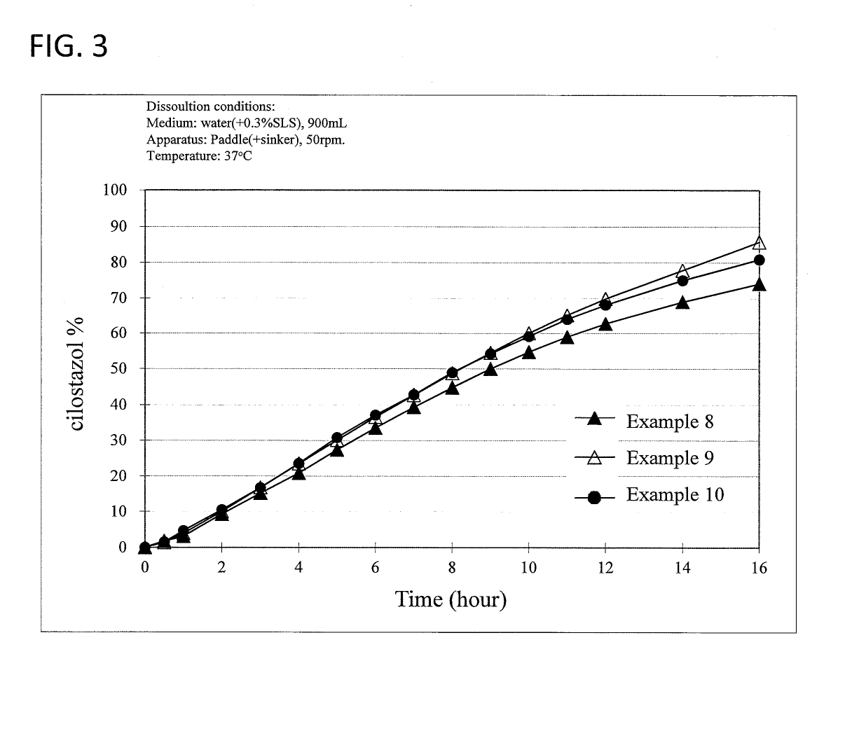

A study was conducted to assess the in vitro dissolution profiles of Examples 8-10. The study was performed according to the procedure described above. Results are shown in Table 7 below and FIG. 3.

TABLE 7
In vitro dissolution profiles of PMR Examples 8-10.

| | % released Cilostazol | | |
|---|---|---|---|
| Time (hour) | Example 8 | Example 9 | Example 10 |
| 0 | 0 | 0 | 0 |
| 0.5 | 1.75 | 1.42 | 1.49 |
| 1 | 3.13 | 3.92 | 4.64 |
| 2 | 9.26 | 10.00 | 10.52 |
| 3 | 15.20 | 16.78 | 16.74 |
| 4 | 20.87 | 23.52 | 23.56 |
| 5 | 27.36 | 30.12 | 30.85 |
| 6 | 33.53 | 36.60 | 37.12 |
| 7 | 39.38 | 42.75 | 42.83 |
| 8 | 44.83 | 48.78 | 48.95 |
| 9 | 50.01 | 54.48 | 54.26 |
| 10 | 54.72 | 60.02 | 59.10 |
| 11 | 58.95 | 65.15 | 63.97 |
| 12 | 62.72 | 69.92 | 68.13 |
| 14 | 69.01 | 77.87 | 75.08 |
| 16 | 74.13 | 85.75 | 80.90 |

The results show that varying amounts of Povidone products used in preparation does not impact the in vitro dissolution profile of a PMR tablet.

### Examples 11-14

### Extended-Release Tablets of Cilostazol and their In Vitro Dissolution Profiles

PMR Examples 11-14, each containing particulate cilostazol in the amount of 200 mg, were prepared from the ingredients shown in Table 8 below following workflow Model 3 described in Table 1. Note that different compressing force was applied when tableting Examples 11-14.

TABLE 8
Compositions for PMR Examples 11-14.

| | Example | Example | Example | Example |
|---|---|---|---|---|
| Ingredients | 11 (mg) | 12 (mg) | 13 (mg) | 14 (mg) |
| Tableting | 18 kg/cm² | 21 kg/cm² | 15.5 kg/cm² | 25 kg/cm² |
| Compressing | | | | |
| Force | | | | |
| Cilostazol | 200 | 200 | 200 | 200 |
| D(0.9) <20 μm | | | | |
| Lactose anhydrous | 160 | 160 | 160 | 160 |
| HPMC K100M | 30 | 30 | 30 | 30 |
| Ethyl cellulose | 330 | 330 | 198 | 198 |
| Stearic acid | 14 | 14 | 14 | 14 |
| Total | 734 | 734 | 602 | 602 |

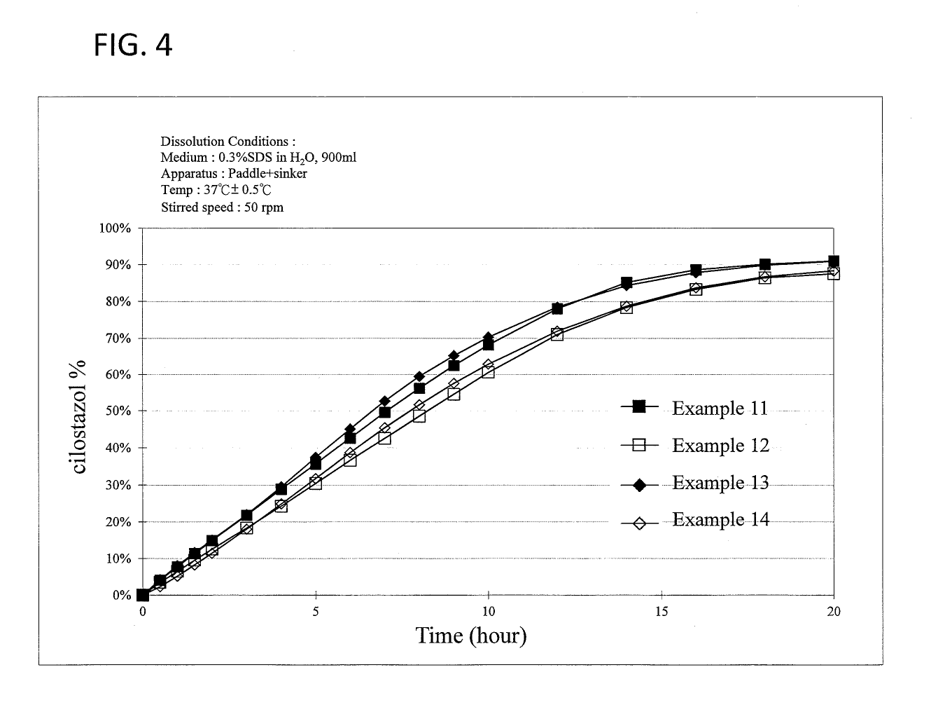

A study was conducted to assess the in vitro dissolution profiles of Examples 11-14. The study was performed according to the procedure described above. Results are shown in Table 9 below and FIG. 4.

TABLE 9
In vitro dissolution profiles of PMR Examples 11-14.

| | % released Cilostazol | | | |
|---|---|---|---|---|
| | Example 11 | Example 12 | Example 13 | Example 14 |
| Time (hour) | (18 kg/cm²) | (21 kg/cm²) | (15.5 kg/cm²) | (25 kg/cm²) |
| 0 | 0 | 0 | 0 | 0 |
| 0.5 | 4.1 | 3.5 | 4.3 | 2.3 |
| 1 | 7.8 | 6.5 | 8.1 | 5.2 |
| 1.5 | 11.4 | 9.6 | 11.6 | 8.3 |
| 2 | 14.9 | 12.5 | 15.0 | 11.4 |
| 3 | 21.8 | 18.3 | 22.0 | 18.0 |
| 4 | 28.9 | 24.3 | 29.5 | 24.9 |
| 5 | 35.7 | 30.4 | 37.4 | 31.7 |
| 6 | 42.6 | 36.6 | 45.1 | 38.7 |
| 7 | 49.5 | 42.6 | 52.6 | 45.4 |
| 8 | 56.1 | 48.5 | 59.5 | 51.6 |
| 9 | 62.5 | 54.5 | 65.3 | 57.5 |
| 10 | 68.2 | 60.6 | 70.3 | 62.9 |
| 12 | 78.0 | 71.0 | 78.5 | 71.9 |
| 14 | 85.2 | 78.3 | 84.4 | 78.7 |
| 16 | 88.6 | 83.3 | 87.9 | 83.8 |
| 18 | 90.1 | 86.4 | 89.8 | 86.7 |
| 20 | 91.0 | 87.6 | 90.9 | 88.3 |

The results show that using a high amount of ethylcellulose, as in Examples 11 and 12, and applying compressing force during tableting, as in Examples 12 and 14, impacted the in vitro dissolution profile of a PMR tablet.

### Examples 15 and 16

### Extended-Release Tablets of Cilostazol and their In Vivo Plasma Profiles

PMR Examples 15 and 16, containing particulate cilostazol in the amount of 100 mg and 200 mg, respectively, were prepared from the ingredients shown in Table 10 below following workflow Model 3 described in Table 1.

TABLE 10
Compositions for PMR Examples 15 and 16

| | Example | Example |
|---|---|---|
| Ingredients | 15 (mg) | 16 (mg) |
| Cilostazol D(0.9) <20 μm | 100 | 200 |
| Lactose anhydrous | 80 | 160 |
| HPMC K100M | 15 | 30 |
| Ethyl cellulose | 132 | 264 |
| Stearic acid | 7 | 14 |
| Total | 334 | 668 |

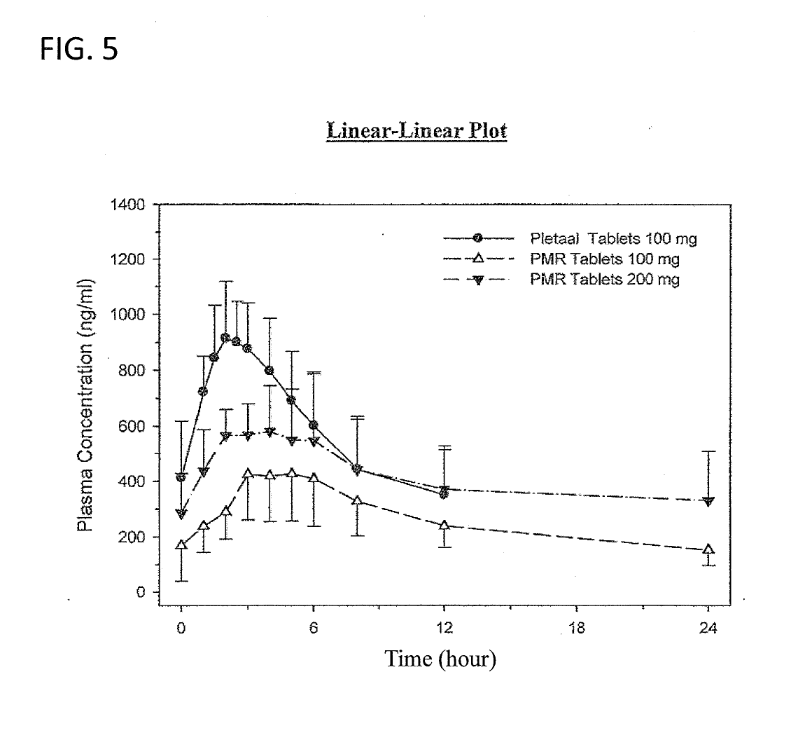

A PK study was conducted, in healthy adult human subjects after a single dose of PMR Example 15 or 16, to assess the in vivo plasma profiles. In addition, Pletaal, an immediate-release tablet, was used as a control in this study. Pletaal in the amount of 100 mg, Example 15, and Example 16 are designated as “Pletaal 100 mg,” “PMR 100 mg,” and “PMR 200 mg,” respectively. Results are shown in Table 11 below and FIG. 5.

TABLE 11
In vivo plasma profiles of PMR Examples 15 and 16, and a Pletaal
tablet, at 0-24 hours after a single dose administration

| | | Example 15 | Example 16 |
|---|---|---|---|
| | Pletaal 100 mg | PMR 100 mg | PMR 200 mg |
| Time (hour) | (ng/mL) | (ng/mL) | (ng/mL) |
| 0 | 412 | 169 | 287 |
| 1.0 | 722 | 239 | 438 |
| 1.5 | 844 | — | — |
| 2.0 | 914 | 289 | 566 |
| 2.5 | 899 | — | — |
| 3.0 | 877 | 425 | 568 |
| 4.0 | 796 | 420 | 581 |
| 5.0 | 690 | 427 | 550 |
| 6.0 | 601 | 409 | 547 |
| 8.0 | 444 | 327 | 442 |
| 12 | 350 | 239 | 371 |
| 24 | — | 152 | 332 |

From the same PK study, Cₘₐₓ, Tₘₐₓ (time required to reach the maximum plasma concentration), and AUC of these three samples are shown in Table 12 below.

TABLE 12
Results of a PK study on PMR Examples
15 and 16, and a Pletaal tablet

| | Cₘₐₓ (ng/ml) | Tₘₐₓ (hr) | AUC (ng*hr/ml) |
|---|---|---|---|
| Pletaal 100 mg | 982 ± 188 | 2.4 ± 1.0 | 7114 ± 1712 |
| PMR 100 mg (Example 15) | 468 ± 189 | 4.5 ± 1.4 | 6236 ± 1640 |
| PMR 200 mg (Example 16) | 636 ± 163 | 3.3 ± 2.1 | 9885 ± 3489 |

The results demonstrate that “PMR 100 mg” (Examples 15) and “PMR 200 mg” (Examples 16) each exhibited an in vivo plasma profile superior to that of “Pletaal 100 mg.” Namely, “PMR 100 mg” and “PMR 200 mg” each showed a Cₘₐₓ much lower than that of “Pletaal 100 mg” (468±189 and 636±163 vs. 982±188) and a Tₘₐₓ much higher than that of “Pletaal 100 mg” (4.5±1.4 and 3.3±2.1 vs. 2.4±1.0).

### Example 17

### An Extended-Release Tablet of Cilostazol and its Efficacy Study

PMR Example 17, containing particulate cilostazol in the amount of 200 mg, was prepared from the ingredients shown in Table 13 below following workflow Model 4 described in Table 1.

TABLE 13
Compositions for PMR Example 17

| | Example |
|---|---|
| Ingredients | 17 (mg) |
| Cilostazol D(0.9) <20 μm | 200 |
| Lactose anhydrous | 160 |
| HPMC K100M | 30 |
| Ethyl cellulose | 264 |
| Stearic acid | 7 |
| Total | 661 |

In a randomized, double-blind clinical trial, Example 17 tablets (PMR 200 mg, once daily) and Pletaal tablets (Pletaal 100 mg, twice daily) were each used to treat a group of 10 peripheral vascular disease patients separately for 16 weeks. At the beginning (0 week), the middle (8 weeks), and the end (16 weeks) of the trial, initial claudication distances (ICDs) were measured from these 20 patients. ICD improvements as a result of treatment were measured as “increase %,” compared to ICDs at the beginning of the trial. Results, summarized in Table 14 below, compare the efficacy of “PMR 200 mg,” i.e., Example 17, and “Pletaal 100 mg” in treating intermittent claudication.

TABLE 14
An efficacy comparison between “PMR 200 mg” (once daily)
and “Pletaal 100 mg” (twice daily)

| | PMR 200 mg, once daily | | Pletaal 100 mg, twice daily | |
|---|---|---|---|---|
| | (n = 10) | | (n = 10) | |
| | Claudication | | Claudication | |
| Time (week) | distance (m) | increase % | distance (m) | increase % |
| 0 | 91.9 | — | 120.3 | — |
| 8 | 125.7 | 45.9% | 217.3 | 69.4% |
| 16 | 233.2 | 103.7% | 218.7 | 69.1% |

The results show that, at the end of 16-week trial, “PMR 200 mg,” i.e., an extended release pharmaceutical composition of this invention, unexpectedly, yielded a much greater clinical improvement (103.7% vs. 69.1%) in treating intermittent claudication than “Pletaal 100 mg,” an immediate-release tablet, even though “PMR 200 mg” was administered once daily and “Pletaal 100 mg” twice daily.

In the same clinical trial, “PMR 200 mg,” unexpectedly, showed much fewer side effects than “Pletaal 100 mg.” Specifically, much more patients reported no adverse event in the “PMR 200 mg” group than those in the “Pletaal 100 mg” group (20% vs. 10%); and much fewer patients reported drug-related adverse events in the “PMR 200 mg” group than those in the “Pletaal 100 mg” group (30% vs. 40%).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A pharmaceutical composition comprising:
particulate cilostazol or a salt thereof having a 90% particle size in a cumulative particle size distribution of 5 to 75 μm;
a cellulose;
a diluent; and
a lubricant,
wherein the pharmaceutical composition is in solid form, includes 15% to 70% the particulate cilostazol or salt thereof by weight, and features an in vivo plasma profile for cilostazol of C_{24 h}/Cₘₐₓ>0.25.

2. The pharmaceutical composition of claim 1, wherein the particulate cilostazol or salt thereof has a 90% particle size in a cumulative particle size distribution of 10 to 30 μm.

3. The pharmaceutical composition of claim 2, wherein the cellulose is hydroxypropylmethylcellulose (HPMC), hydroxyethyl cellulose, hydroxyethylmethyl cellulose, hydroxypropyl cellulose, ethylcellulose, cellulose acetate phthalate, cellulose acetate, methylcellulose, hypromellose phthalate, or a combination thereof.

4. The pharmaceutical composition of claim 2, wherein the diluent is calcium carbonate, calcium phosphate, calcium sulfate, dextrates, dextrose, erythritol, fructose, kaolin, lactitol, lactose, mannitol, simethicone, sodium chloride, sorbitol, starch, sucrose, sulfobutylether-β-cyclodextrin, trehalose, xylitol, microcrystalline cellulose, or a combination thereof.

5. The pharmaceutical composition of claim 2, wherein the lubricant is calcium stearate, glycerin monostearate, glyceryl behenate, glyceryl palmitostearate, hydrogenated castor oil, hydrogenated vegetable oil, light mineral oil, sodium lauryl sulfate, sodium stearyl fumarate, stearic acid, zinc stearate, sodium benzoate, magnesium stearate, myristic acid, palmitic acid, poloxamer, polyethylene glycol, potassium benzoate, talc, or a combination thereof.

6. The pharmaceutical composition of claim 3, wherein the cellulose is HPMC, ethylcellulose, or a combination thereof.

7. The pharmaceutical composition of claim 4, wherein the diluent is lactose, microcrystalline cellulose, or a combination thereof.

8. The pharmaceutical composition of claim 5, wherein the lubricant is magnesium stearate, stearic acid, or a combination thereof.

9. The pharmaceutical composition of claim 2, wherein the cellulose is HPMC, ethylcellulose, or a combination thereof; the diluent is lactose, microcrystalline cellulose, or a combination thereof; and the lubricant is magnesium stearate, stearic acid, or a combination thereof.

10. The pharmaceutical composition of claim 1, wherein the pharmaceutical composition features the area under curve for cilostazol of 4600-13400 ng*hr/ml.

11. The pharmaceutical composition of claim 1, wherein the Cₘₐₓ is 280-800 ng/ml.

12. The pharmaceutical composition of claim 1, wherein the C_{24 h} is >0.1 μg/ml.

13. The pharmaceutical composition of claim 10, wherein the Cₘₐₓ is 280-800 ng/ml and the C_{24 h} is >0.1 μg/ml.

14. The pharmaceutical composition of claim 13, wherein the amount of particulate cilostazol or salt thereof is 100 mg.

15. The pharmaceutical composition of claim 13, wherein the amount of cilostazol or a salt thereof is 200 mg.

16. The pharmaceutical composition of claim 14, wherein the Cmax is 280-660 ng/ml and the area under curve is 4600-7900 ng*hr/ml.

17. The pharmaceutical composition of claim 15, wherein the Cmax is 470-800 ng/ml and the area under curve of 6400-13400 ng*hr/ml.

18. A method of preparing the pharmaceutical composition of claim 1, the method comprising:
mixing particulate cilostazol or a salt thereof having a 90% particle size in a cumulative particle size distribution of 5 to 75 μm, a first cellulose, a diluent, and water to form a homogenous mixture;
granulating the homogenous mixture to form granules;
heating the granules to form dried granules;
mixing the dried granules, a lubricant, and optionally a second cellulose to form a blend; and
compressing the blend to form tablets.

19. The method of claim 18, wherein the first cellulose is HPMC, hydroxyethyl cellulose, hydroxyethylmethyl cellulose, hydroxypropyl cellulose, ethylcellulose, cellulose acetate phthalate, cellulose acetate, methylcellulose, hypromellose phthalate, or a combination thereof.

20. The method of claim 18, wherein the diluent is calcium carbonate, calcium phosphate, calcium sulfate, dextrates, dextrose, erythritol, fructose, kaolin, lactitol, lactose, mannitol, simethicone, sodium chloride, sorbitol, starch, sucrose, sulfobutylether-β-cyclodextrin, trehalose, xylitol, microcrystalline cellulose, or a combination thereof.

21. The method of claim 18, the lubricant is calcium stearate, glycerin monostearate, glyceryl behenate, glyceryl palmitostearate, hydrogenated castor oil, hydrogenated vegetable oil, light mineral oil, sodium lauryl sulfate, sodium stearyl fumarate, stearic acid, zinc stearate, sodium benzoate, magnesium stearate, myristic acid, palmitic acid, poloxamer, polyethylene glycol, potassium benzoate, talc, or a combination thereof.

22. The method of claim 18, wherein the second cellulose is ethylcellulose, cellulose acetate phthalate, cellulose acetate, methylcellulose, hypromellose phthalate, or a combination thereof.

23. The method of claim 19, wherein the diluent is calcium carbonate, calcium phosphate, calcium sulfate, dextrates, dextrose, erythritol, fructose, kaolin, lactitol, lactose, mannitol, simethicone, sodium chloride, sorbitol, starch, sucrose, sulfobutylether-β-cyclodextrin, trehalose, xylitol, microcrystalline cellulose, or a combination thereof; the lubricant is calcium stearate, glycerin monostearate, glyceryl behenate, glyceryl palmitostearate, hydrogenated castor oil, hydrogenated vegetable oil, light mineral oil, sodium lauryl sulfate, sodium stearyl fumarate, stearic acid, zinc stearate, sodium benzoate, magnesium stearate, myristic acid, palmitic acid, poloxamer, polyethylene glycol, potassium benzoate, talc, or a combination thereof; and the second cellulose is ethylcellulose, cellulose acetate phthalate, cellulose acetate, methylcellulose, hypromellose phthalate, or a combination thereof.
